(19) United States
(12) Patent Application Publication
(10) Pub. No.: US 2005/0034725 A1
(43) Pub. Date: Feb. 17, 2005

(54) Lung-ventilating arrangement

(76) Inventors: Stefan Stromberg, Sigtuna (SE); Nils Olof Eriksson, Enskede (SE)

Correspondence Address: Steven P Schad;Banner & Witcoff, Eleventh Floor, 1001 G Street NW, Washington, DC, 20001-4597, US

(21) Appl. No.: 10/481,459

(22) PCT Filed: Jun. 17, 2002
(86) PCT No.: PCT/SE02/01168

(30) Foreign Application Priority Data
Jun. 19, 2001 (SE) 0102172-4

(51) Int. Cl. A61M 16/00; A62B 7/00

(52) U.S. Cl. 128/205.13; 128/204.18

(57) ABSTRACT

The invention relates to a respiratory arrangement (1) that includes: a control unit (3), a plurality of gas delivery hoses (4), and a patient-allocated or -related device (5), such as a facemask, wherein first end portions (4a) of respective hose face towards the control unit while second end portions (4b) of respective hose face towards said device. The control unit (3) inlcudes means for passing a gas flow, intended for inhalation, or passing a gas flow. intended for insufflation (“1”) to said patient via a first hose (4c). An expiration valve (6), situated close to the patient-allocated or -related device is adapted to release a gas flow intended for expiration (“E”) to the free surroundings and said expiration valve can be switched to one of two settings, via a second (4d) of said gas delivery hoses. A container (7) or gab containing a reserve volume (7′) is adapted to generate a gas flow, which is intended for manual insufflation and which can be actuated by pressurising the container (7) or bag temporarily and for or during a short period of time. The reserve volume (7′) of the pressurised container (7) or bag is delivered to the first hose (4c), via a valve arrangement (41), and said pressure is delivered to the second hose, via said valve arrangement (41) to cause the expiration valve (6) to take a closed state, associated with a manufal insufflation phase.

### FIELD OF INVENTION

The present invention relates generally to a lung ventilating arrangement and more specifically to a lung ventilating arrangement of the kind that includes at least one lung ventilator control unit, a number of gas delivery lines or hoses and a patient-allocated or patient-related device.

The control unit is normally adapted to achieve a “mechanical” ventilation of the lungs, which, in principle, is based on an insufflation gas delivery to a patient in a controllable manner and in chosen periods or cycles, associated with and adapted for a normal inhalation cycles.

Gas delivery lines are normally comprised of relatively long hose sections, each connected to different shorter channels. However, each of these lines will be referred to hereinafter as a “gas delivery hose” for the sake of simplicity.

More particularly, the present invention relates to an application in lung ventilators that can be switched between at least two functional modes.

A first of these functional modes is designated “Spontaneous/Manual” and a second of said modes is designated “CMV” (Controlled Mechanical Ventilation).

In the case of an application, described in more detail hereinafter, by gas delivery hose is meant a hose, which is used partly in a proposed application, partly in a proposed embodiment, and partly beneficially to deliver inhalation gas or insufflation gas or inhalation air or insufflation air or a mixture of air and some other gas, such as oxygen gas, wherein said hose has a relatively large cross-sectional area and can be used to control an expiration valve, such as to cause the valve to take or switch over to one of said two states. The term also refers to one or more additional hoses, for measuring different criteria.

In the case of the inventive lung ventilating arrangement, first ends of said hoses shall face towards the control unit or the ventilator, whereas the other ends of said hoses shall face towards a patient-allocated or -related device and co-act with the ventilator and said patient-allocated device in a known manner.

One of these gas delivery hoses, referred to hereinafter as the first hose, functions to pass or lead to the patient a gas flow, intended for and adapted for a spontaneous inhalation, or to drive or under overpressure press, from the ventilator, a gas flow intended for mechanical insufflation.

It is also possible to use a valve, which is situated close to the patient-allocated or -related device, which can be adapted to allow exhalation gas to flow to the free surroundings.

This expiration or exhalation valve can be controlled from the control unit into one of its two positional settings. This control may be effected beneficially by pressurising a second of said gas delivery hoses, this hose being referred to hereinafter as the second hose.

It is expected that the inventive lung ventilating arrangement will find special application in respect to patients that are under treatment and that are offered pain relief or the like via intravenous anaesthesia.

More particularly, the invention is intended to provide benefits, during anaesthesia and recovery phases, during which there may be a reason to temporarily support a spontaneous breathing in the chosen functional mode “Spontaneous/Manual”, by manually initiating and carrying out an insufflation phase.

A system, in which the present invention is intended to find a particularly suitable application, is known to the art and described in a Swedish Patent Application, allotted the Serial Number 99 02051-3 (Corresponding to an International Patent Application Serial Number PCT/SE 00/01067, published under number WO-A1-00/74757).

### DESCRIPTION OF THE BACKGROUND ART

A large number of designs related to lung ventilating arrangements of the aforedescribed kind are known to the art and normally comprise a lung ventilator unit, with an associated control unit.

Present-day control units are relatively complicated, with regard to detecting and controlling different patient-related criteria, and it is not unusual to “customize” the functions in the lung ventilator unit to choose application areas and conditions in other respects.

Lung ventilator units, that include such control units, can normally be set to one of a number of available function modes.

The present invention finds its application when the lung ventilator unit can be set to one of a number of available function modes, herein referred to as the “Spontaneous/Manual” mode.

This function mode takes, as its starting point, the assumption that the patient can breathe spontaneously, with periodic inhalation and expiration phases, and on the assumption that, with respect to the well-keeping of the patient, he/she will require additional measures, namely a manual administration of an insufflation phase on different occasions.

There is provided to this end a flexible container or bag, that is filled with gas, such as air, wherewith the air is administered manually, via the insulation phase, by manually compressing the container or bag and therewith forcing out an enclosed reserve volume, either completely or partially.

Lung ventilating arrangements, of the kind intended here, are also provided with an additional functional mode, designated “CMV”, in which the patient is administered successive insufflation phases mechanically, via a control unit.

An earlier known arrangement of the aforesaid kind is constructed to enable a first chosen functional mode to be used on patients for the general purpose of ventilating the lungs of a patient, who breathes spontaneously, and to be used via a second choice of functional mode on patients that are in a more or less deep anaesthetised state.

In the case of known arrangements that include a lung ventilating unit or lung ventilator there is used a number, at least two, gas delivery hoses and a patient related device and a device-allocated or -related valve.

There is proposed in this respect the use of a first gas delivery hose, which extends between the lung ventilator and the patient and which is adapted to lead a gas or air, intended for spontaneous inhalation or to administer to the patient gas or a gas mixture intended for insufflation mechanically and/or manually, with the first hose connected to a device allocated to and close to said patient and including an expiration valve connected to the lung ventilator.

Reference is made to the contents of Swedish Patent Application No. 99 02051-3 in this regard.

The patient connected or allocated device may have different forms, such as a facemask, a tracheal tube or larynx mask.

It is also known, with respect to the use of lung ventilating units on living creatures in a non-anaesthetised state, for instance from Patent Publication U.S. Pat. No. 3,961,627, to include in the flow direction of the gas and in the proximity of the living creature, or patient, a control valve and a measuring device, including a requisite sensor unit that functions to determine the rate of flow and the pressure of said gas.

The valve driving mechanism is comprised, in this case, of a servomotor that receives control signals from a valve-control unit.

Also known to the art is a control unit, which includes the necessary means for evaluating therapeutic and/or diagnostic criteria and which is of the nature described and illustrated in more detail in Swedish Patent Application 99 01688-3 (corresponding to International Patent Application No. PCT/SE 00/00910, with Publication No. WO-A1-00/67820).

It is also known to allow an insulation gas either to consist of pure air or air mixed with oxygen gas to a greater or lesser extent, or solely oxygen gas and/or additives.

Consequently, a number of hoses have been mentioned in the following description as “gas delivering hoses”, regardless of whether gas flows through the hose or whether it is used solely to pressurise an expiration valve, in order to close said valve.

### SUMMARY OF THE INVENTION

Technical Problems

When taking into consideration the technical deliberations that a person skilled in this particular art must make in order to provide a solution to one or more technical problems that he/she encounters, it will be seen that on the one hand it is necessary initially to realise the measures and/or the sequence of measures that must be undertaken to this end, and on the other hand to realise which means is/are required in solving one or more of said problems. On this basis, it will be evident that the technical problems listed below are highly relevant to the development of the present invention.

With respect to a lung ventilating arrangement, that includes a lung ventilating unit and a control unit belonging to a lung ventilator, and also a number of gas delivery hoses and a patient-allocated or -related device, where one ore first ends of the hoses face towards the ventilator unit or control unit while the other ends of the hoses face towards the patient or the patient-allocated or -related device, wherein said control unit shall include means for allowing a gas flow for spontaneous inhalation and/or a gas flow intended for mechanical insufflation to be delivered to the patient via one of said hoses, and wherein an expiration valve situated close to or adjacent to the patient-allocated or -related device shall be adapted to allow the gas flow belonging to the expiration phase to pass to the free surroundings, and wherein the expiration valve can be controlled by the control unit such as to cause the valve to take one of two settings, by pressurising another of said gas delivery hoses, it will be seen that a technical problem exists in the provisions of conditions whereby during a first functional mode, in which the patient is expected to be able to breathe spontaneously in and out, a brief manual insufflation phase or the delivery of an insufflation-related gas flow can be effected by emptying a flexible reserve container or bag, either completely or partially, and also create conditions such that the system will be prevented from being controlled to values that exhibit dangerous overpressures during such an insufflation phase, with the aid of a valve arrangement (not serving as solely an overpressure valve).

A technical problem also resides in the ability to realise the significance of and the advantages associated with creating manually to this end an insufflation phase, in which the functional mode chosen is the “Spontaneous/Manual” mode, and allowing the use of such a valve arrangement, in which the displacement of a piston between two end states can take place in response to a gas flow acting on said piston and generated from a manually pressurised reserve volume contained in a flexible container or bag.

There then exists a technical problem in the ability to realise the significance of and the advantages that are associated with allowing the reserve volume of said container or bag to be delivered as a gas flow to the end portion of the first hose, by exerting an overpressure to the container or bag manually, and feeding said gas flow through the hose to the other end portion thereof and to the patient as an insufflation phase with the aid of the special valve arrangement, that has been constructed to provide a plurality of different valve functions via one and the same piston movement.

Another technical problem resides in the ability to realise the significance of and the advantages associated with allowing the manually generated overpressure to be applied to a second of said gas delivery hoses, also via a valve function in said valve arrangement, so as to cause said expiration valve to take a closed state, adapted for the initiated and performed insufflation phase.

Another technical problem resides in the ability to realise the significance of and the advantages that are associated with the ability to influence a membrane, included in said expiration valve, with a “static” pressure on one side of the membrane from the overpressure generated in the flexible reserve container while using said valve arrangement, and to allow the other side of the membrane to be influenced with a lower flow-related pressure, caused by the flow of gas through said first hose.

Another technical problem resides in the ability to realise the significance of and the advantages that are afforded by allowing a chosen pneumatic resistance value, in the pneumatic circuit that includes the first hose to, cause an adapted pressure reduction, via the flow generated in the first gas delivery hose, and thereby create a pressure difference, acting across the mutually opposite pressure-loaded surfaces of the membrane, that is required to hold the expiration valve closed during the manual expiration phase.

Another technical problem resides in the ability to realise the significance of and the advantages that are afforded by allowing the expiration valve to include a flexible membrane, whose pressure-actuated surface, that faces a first hose, is smaller than the pressure actuated surface, that is influenced by the static overpressure in said second hose.

There then exists a technical problem in the ability to realise the significance of and the advantages afforded by allowing said expiration valve to include a flexible membrane, that is active against a valve seat, whose one surface, that faces the first hose, is smaller than a second surface, that faces the second hose and/or a cavity belonging to the expiration valve.

Another technical problem resides in the ability to realise the significance of and the advantages that are afforded by allowing the expiration valve to be connected to the patient-allocated or -related device or to have said device to be integral therewith.

Another technical problem resides in the ability to realise the significance of and the advantages that are afforded by allowing said ventilator and/or its control unit to include a T-coupling, belonging to the first gas delivery hose, with a T-coupling-related valve arrangement, which is allocated a plurality of valve functions through the medium of one single piston movement, wherein the container or bag is connected directly to said valve arrangement.

It will be seen that a technical problem resides in the ability to realise the significance of and the advantages that are afforded by allowing the lung ventilator and/or the control unit to include said valve arrangement, which, in response to an overpressure generated in the container or bag, permits the flow and passage of gas to the patient during an insufflation phase and which valve arrangement and a function mode related valve unit can deflect the same overpressure for “static” actuation of the expiration valve.

Another technical problem resides in the ability to realise the significance of and the advantages that are afforded by allowing the lung ventilator and/or the control unit to include and to carry a function mode related valve unit, which be longs to the second gas delivery hose and which is adapted to allow through-coupling and pressurisation of the second gas delivery hose, so as to close said expiration valve by urging a membrane against a valve seat.

Another technical problem resides in the ability to realise the significance of and the advantages that are afforded by allowing one of the valve functions in the valve arrangement to be adapted to permit passage of insufflation gas to the container or bag in a set state.

Yet another technical problem resides in the ability to realise the significance of and the advantages that are afforded by allowing at least a first valve function and a second valve function, and preferably also further valve functions, to be incorporated in one single valve arrangement.

Solution

The present invention is thus based on a lung ventilating arrangement that includes a lung ventilator which includes a control unit, wherein said arrangement further includes a number of gas delivery hoses, connected to the lung ventilator and a patient-allocated or patent-related device, for conducting gas to the patient for spontaneous inhalation and to enable gas intended for insufflation to be delivered to the patient.

One or first end parts of respective hoses shall face towards the lung ventilator or the control unit, and the other or second end parts of said respective hoses shall face towards the patient-allocated or -related device and be secured to the control unit and to the patient-allocated or -related device respectively in a known manner.

The control unit shall include means for allowing said gas flow, intended for inhalation or insufflation, to be delivered to the patient via one of the hoses, a first hose, wherein the expiration valve, situated close to or adjacent to the patient-allocated or -related device is adapted to release the expiration gas flow to the free surroundings and to be controllable to one of two settings, with the aid of said expiration valve and via a second of said gas delivery hoses.

With the intention of solving one or more of the aforesaid technical problems, the present invention is based upon and builds on the use of a flexible container or bag, that holds a reserve volume of gas adapted for a manual insufflation phase of a short duration, wherein said flexible container or bag can be pressurised manually for a short period of time.

That part of the reserve volume of the container or bag, which converts to a gas flow via said pressurisation of the flexible container or bag as a result of said applied pressure and compression of the container or bag, shall be delivered to the patient through a valve arrangement and said first hose as an insufflation phase, although said overpressure shall also be applied or deflected to the second of said gas delivery hoses, so that said expiration valve can be set to a closed insufflation-allocated or -related state.

By way of proposed embodiments, that lie within the scope of the inventive concept, it is proposed that the expiration valve shall include an elastically flexible membrane, whose one pressure-actuated surface, facing a first hose, is smaller than a second pressure-actuated surface, that can be influenced directly by an overpressure prevailing in a second hose.

It is also proposed that said expiration valve shall include a membrane, whose one surface, facing said first hose. is smaller than a second surface, facing a cavity in the expiration valve.

It is also proposed that the expiration valve is connected to the patient-allocated or -related device or that said device is integral therewith.

It is also proposed, in accordance with the invention, that the lung ventilator and/or a control unit shall include a T-coupling and/or a valve arrangement belonging to the first hose, said T-coupling and/or said valve arrangement being connected directly to the container or bag.

Furthermore, the control unit includes or co-acts with a special valve arrangement, that is actuable by the gas flow from the container or bag, caused via the overpressure exerted on the reserve volume, wherein, when in a set state, the valve arrangement allows insufflation gas to pass to the patient and, in addition, deflects an overpressure to the expiration valve in the absence of any appreciable gas flow.

It is also proposed that the control unit may include or at least co-act with a valve unit, which is separate from the valve arrangement, and which belongs to or is allocated to said second gas delivery hose, wherein said valve unit is adapted to pressurise the second hose in a function mode related fashion such as to close said expiration valve during the mechanical or manual insufflation phase.

The valve arrangement also includes a valve function, which, in a “Spontaneous/Manual” state, can permit insufflation gas fed from the lung ventilator to pass to the container.

It is also proposed that a first valve function co-acts with a second valve function, wherein both functions can be considered to be included in a T-coupling or in said valve arrangement.

Advantages

Those advantages primarily associated with an inventive lung ventilating arrangement reside in the creation of conditions in which, at least when the control unit of the lung ventilator is set to a state corresponding to a first function mode “Spontaneous/Manual”, there can be generated an insufflation phase of short duration by pressurising a flexible container or bag, wherein the gas flow thus generated directly actuates a valve arrangement.

Also created are conditions in which gas is allowed to flow to a first delivery hose and to said patient, via said valve arrangement, not solely during an overpressure and a manual insufflation phase, but also to create an overpressure to a second of said gas delivery hoses, via said valve arrangement, such as to forcibly cause an expiration valve close to a patient to take a closed state, assigned to said insufflation phase.

The valve arrangement can thus be actuated from a first to a chosen second state via the gas flow generated, wherewith the valve arrangement returns to its first state when the gas flow ceases.

Another advantage is that when all or some of the reserve volume has been used and ejected from the flexible container or bag, a valve function, included in the valve arrangement, allows that part of the reserve volume, which has been used, to be replenished from the ventilator.

The primary characteristic features of an inventive lung valve arrangement are set forth in the characterising clause of the accompanying claim 1.

### BRIEF DESCRIPTION OF THE DRAWINGS

A lung ventilating arrangement at present preferred and having features characteristic of the present invention will now be described in more detail with reference to the accompanying drawings, in which;

FIG. 1 is a schematic illustration of a lung ventilating arrangement, with which a lung ventilator and/or control unit has been supplemented in accordance with the present invention;

FIG. 2 is a perspective section view of a valve, which is designated an expiration valve and which is situated close to or adjacent to a patient;

FIG. 3 is a highly simplified and more schematic view, illustrating significant function parts of the inventive arrangement in a control unit for providing the temporary supply of a percentage of insufflation gas via a manual insufflation phase during the function mode “Spontaneous/Manual”, said temporary supply being associated with the present invention;

FIG. 4 is a principle diagram of a lung ventilating unit and/or a control unit, in which a number of invention-related and necessary functions are included, such as to provide the significant characteristic features of the present invention;

FIG. 5 illustrates a control unit-actuated valve arrangement in a state in which the reserve volume of the container or bag is depleted via an overpressure, applied to the container or bag, via a gas flow generated thereby and used as and in a manual insufflation phase;

FIG. 6 illustrates the valve arrangement in a state in which an empty container or bag can be filled successively to its maximum reserve volume, via valve functions in said valve arrangement;

FIG. 7 illustrates an example of means for setting the valve arrangement in two different function module-related states; and

FIG. 8 illustrates part of the valve arrangement, which constitutes a chosen embodiment of a seal between a cylinder-housed piston, which moves up and down in response to gas flow, and an outer part of the cylinder.

### DESCRIPTION OF AN EMBODIMENT AT PRESENT PREFERRED

It is pointed out initially that we have chosen to use in the following description of embodiments at present preferred and including significant characteristic features of the invention and illustrated in the figures of the accompanying drawings special terms and terminology with the intention of illustrating the inventive concept more clearly.

However, it will be noted that the expressions chosen here shall not be seen as limited solely to the chosen terms used in the description, but that each term chosen shall be interpreted as also including all technical equivalents that function in the same or at least essentially the same way so as to achieve the same or essentially the same intention and/or technical effect.

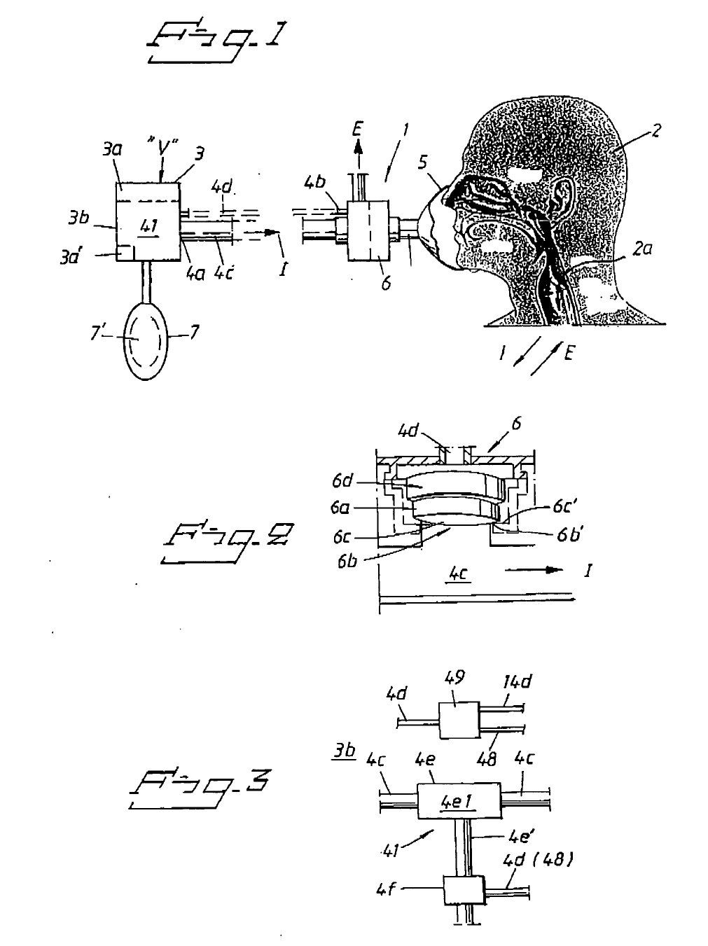

Illustrated schematically in FIG. 1 is a lung ventilating arrangement 1, which includes significant characteristic features of the present invention. The arrangement 1 is coupled or connected to a patient in a known manner, said patient being shown in the form of a person 2, wherein a gas flow, intended for spontaneous inhalation or at least for mechanical insufflation, shall pass through the airways or respiratory tract 1a of said person 2 in a first direction “I” in a known manner and provide an expiration of gas flow from said patient 2 in a second direction “E” opposite to the first direction “I”, said gas flow for said mechanical insufflation being referred to as an insufflation phase.

The lung ventilating arrangement 1 includes a ventilator unit “V” or a lung-ventilating unit 3, which includes a control unit 3a.

Control units 3a of the kind concerned here are complicated mechanical and electronic devices, and hence the following description is concentrated solely on those parts that are directly concerned with the present invention and its function.

The arrangement also includes a number of gas delivery hoses 4 and a patient-allocated or -related and patient-proximate device 5, which has the form of a facemask in the case illustrated.

The arrangement 1 illustrated in FIG. 1 is, to a great extent, illustrated and described in more detail in Swedish Patent Application 99 02051-3.

A valve arrangement 6 is coupled to the device 5 or facemask.

This valve arrangement is described in more detail in Swedish Patent Application 00 04066-7, the contents of which application shall be considered as forming a part of the present application and to constitute a part of the present description.

The present invention requires the use of an expiration valve 6 in the form of a membrane, to be described in more detail.

The present invention will be described with reference to the use of solely two gas-delivering hoses, although a hose bundle may include further hoses.

A first hose 4c is intended for a gas flow, while a second hose is intended to convey an occurring pressure to one side of the membrane in the expiration valve.

First end portions 4a of the hoses 4 face towards the control unit, while second end portions 4b face towards said device, or more precisely towards the expiration valve arrangement 6.

The control unit 3a also includes means for controlling mechanical insufflation phases in accordance with the setting of a function mode selector 3a′.

The function mode selector 3a′ can be set to either one of two function modes, one designated “CMV” and one designated “Spontaneous/Manual”.

The function mode “CMV” creates conditions for passing or urging insufflation phase after insufflation phase periodically to said patient 2 from the control unit 3a and via one of the gas delivery hoses 4c, at a rate that corresponds to a normal breathing rhythm of the patient.

The valve arrangement, in the form of said expiration valve 6, situated close to or adjacent to the patient allocated device 5, is adapted to allow a gas flow intended for expiration to be released periodically to the free surroundings and at an overpressure.

The valve arrangement 6 can be caused to take one of two available positional settings by the control unit 3a, by pressurising a second of said gas delivery hoses, namely the delivery hose 4d in the insufflation phase.

It is an intention of the invention to readily provide a brief and manually initiated and performed insufflation phase under otherwise good premises for spontaneous breathing when the function mode “Spontaneous/Manual” is activated.

Also shown in FIG. 1 is a container 7 or bag, which is assumed to contain a reserve volume 7′, which will generate a gas flow intended for a temporary insufflation phase when said container or bag is subjected manually to a pressure.

More particularly, the present invention shall be put into effect with the aid of said flexible container 7, an elastic or flexible rubber bladder, by temporarily and briefly pressurising the hose 4c for the passage of gas therethrough while, at the same time, increasing the pressure in the hose 4d, in accordance with patient requirements.

More specifically, it is a question of allowing all or some of the reserve volume 7′ of the container 7 or bag to be delivered to the first of said hoses 4c under a manually created overpressure in the flexible container 7 or bag, said first hose being connected directly to said patient 2, and to delivering said generated overpressure to the second of said gas delivery hoses, namely said hose 4d, so as to cause said expiration valve 6 to take a closed state, an insufflation phase associated state.

The gas flow intended for the insufflation phase is referenced “I” while the gas flow intended for the expiration phase is referenced “E”, said phases occurring sequentially after each other.

Shown in FIG. 1 is a valve arrangement 41, which is necessary to the function of the invention and the nature of which will be described in more detail hereinafter.

The valve arrangement will be described with reference to FIG. 2 in the form of the construction and function of an expiration valve 6.

The valve arrangement 6 includes a flexible membrane 6a, which has a pressure actuated surface 6b that faces towards the first gas delivery hose 4c, and a pressure actuated surface 6c that faces towards the second hose 4d.

When the pressure force acting on the surface 6b is greater than the pressure force acting on the surface 6c, the expiration valve 6 will open. Conversely, when the pressure force acting on the surface 6b is lower than the pressure force acting on the surface 6d, the expiration valve 6 will close.

The latter case is most prominent with respect to the present invention.

In accordance with the invention, the rise in pressure obtained by compression of the container 7 or bag, in the absence of any appreciable gas flow, causes the expiration valve 6 to close. This higher pressure propagates through the hose 4d, so as to act on the surface 6c.

Compression of the container 7 or bag also generates a rise in pressure in the hose 4c, this pressure acting on the surface 6b.

The function of the invention depends on the valve arrangement 6 being closed during the period of time when a pressure rise is delivered to the system through the medium of a gas flow.

Since the pressure rise in the hose 4d will act “statically” on the surface 6c in the absence of flow and therewith no pressure losses in the hose 4d, while the pressure rise in the hose 4c will act on the surface 6b via a gas flow and result in pressure losses, there is a favourable condition for the membrane 6a being held in its expiration and closed state.

In addition, it is proposed that the dimensions of the surface 6b shall be smaller than those of the pressure-actuated surface 6c.

It is particularly proposed that said valve arrangement 6 shall include a cupped membrane 6a and that the surface 6b, which faces towards the insufflation hose 4c, is smaller than the surface 6c, which faces towards a valve-associated cavity 6d, this feature being achieved by allowing a peripheral, circular surface section 6b′ of the membrane 6a to rest against a circular valve seat 6e, with the surface 6c extending over said valve seat 6e.

The expiration valve 6 may be connected to the patient-allocated or -related device 5, as shown in FIG. 1, or these components may be integral with one another.

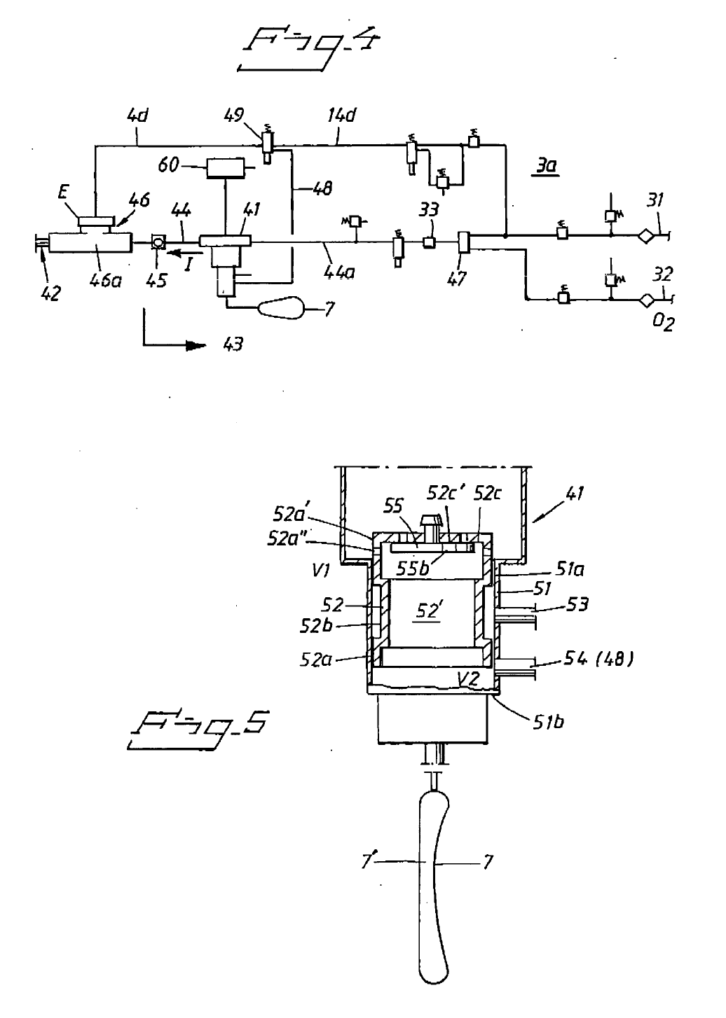

FIG. 3 illustrates schematically functions, units and details that are significant with respect to the conditions afforded by the present invention.

It will be obvious that such a control unit 3a will include far more units and components than are shown. However, these further units and components have not been shown for the sake of clarity.

More particularly, FIG. 3 solely illustrates the principle supplements that will be required to implement the features and functions associated with the invention.

One significant feature in this regard is to make a cut in the first gas delivery hose, the so-called insufflation hose 4c, within the casing of the control unit 3a, or adjacent the casing 3b, and to place in said cut a T-coupling 4e belonging to the insufflation hose 4c.

The T-coupling 4e includes a connecting part 4e′, which is adapted for co-action with the container 7 or bag.

According to the invention, the T-coupling 4e shall include a valve function 4e1, where the function mode “CMV” requires free gas passage through the insufflation hose 4c and where passage of gas to the container 7 is blocked.

The valve function 4e1 shall take the open state shown in FIG. 7 in the case of the function mode “Spontaneous/Manual”, meaning that insufflation gas from the lung ventilator can pass continuously through the hose 4c as above, and that insufflation gas can also replenish the container.

As will be seen from FIG. 3, a coupling device 4f, having a valve function, is used between the connection 4e′ and the container 7 or bag.

This coupling device 4f, or a valve arrangement, requires a valve function that allows insufflation gas to be quickly passed over to the hose 4c from the container 7 or bag, on the one hand, and to provide the function of allowing the reserve volume 7′ to be replenished slowly, on the other hand.

The control unit 3a also includes a coupling device 4f that has a further valve function, belonging to the second gas delivery hose 4d, this valve function being adapted to pressurise the second hose 4d to an extent such that said pressure will close the expiration valve 6 and hold said valve closed during the time period when inhalation assistance is required temporarily, via a manual insufflation phase.

According to the invention, all valve functions shall be capable of being combined into one valve arrangement 41.

An embodiment of a valve arrangement 41 at present proposed and a proposed coupling in the control unit 3a will now be described with reference to FIGS. 4 to 8.

This coupling feature will be evident from the coupling diagram, shown in FIG. 4.

Thus, there is shown the use of an expiration valve 46, which is built into a casing 46a, and connected to the patient 42 and also to a first gas delivery hose, designated an insufflation hose 44.

The expiration valve 46 can be caused to take one of two fixed states, via a gas pressure line 4d.

This line 4d is pressurised via a first valve unit 49, related to a function mode for closing the expiration valve 46 periodically at the function mode “CMV” by periodically pressurising a line 14d.

In this case, the valve unit 49 takes a state, corresponding to the function mode “CMV”, and shuts out any variation in pressure that may possibly occur on a line 48 from the valve arrangement 41.

The valve 49 can also take a “Spontaneous/Manual” state, wherewith solely pressure variations occurring on line 48 are delivered to line 4d and to the expiration valve 6.

When necessary, the gas delivery hose 44 includes a check valve 45 so as to be able to conduct inhalation gas and, when necessary, insufflation gas during the manual insufflation phase, from the control unit 3a and a valve arrangement 41 included in said unit and then to the patient 42, by creating in this way an adapted opening resistance when the pneumatic system so requires, in order to achieve the inventive function.

The check valve 45 may conveniently be incorporated in the valve arrangement 41, although this embodiment has not been shown in detail.

The hose 44 is coupled to the valve arrangement 41 in a known manner, said arrangement being connected to an air supply 31 and to an oxygen-gas supply 32, via a line 44a.

Requisite mixing of the air and oxygen-gas is achieved in a mixing valve 47.

In function mode “CMV”, insufflation gas is delivered periodically for mechanical ventilation, while in function mode “Spontaneous/Manual” there is a continuous delivery of insufflation gas.

More particularly, the invention proposes the use of a needle valve 33, for regulating the flow of gas from the lung ventilator “V” (43) during the function mode “CMV” in a known manner, and above all during the function mode “Spontaneous/Manual” in accordance with the recommendations in respect of the invention, where a purely constant flow of gas is concerned.

In this respect, the gas flow shall be adapted to at least satisfy the need of the patient 2 in respect of spontaneous breathing, and to provide replenishment of the container 7 or bag within a chosen time period, and it is necessary to allow the adapted gas flow to be delivered in excess.

This excess will pass through the expiration valve 6.

This setting serves the purpose of enabling the container 7 or bag to be replenished against the action of a pneumatic resistance prevailing within the system, such as patient resistance, the resistance of the expiration valve and/or the effect of the opening resistance of the check valve 45.

The regulated continuous flow through the needle valve 33 and a well-tuned resistance relationship within the system are responsible for enabling the container 7 or bag to be replenished and pressurised in a chosen and adapted manner.

The construction of the proposed valve arrangement 41 will now be described in detail with reference to FIGS. 5 through 8.

The valve arrangement 41 includes a piston 52, that can move freely up and down in a cylinder 51.

The function requires a seal between the piston 52 and an inner surface 51a of the cylinder.

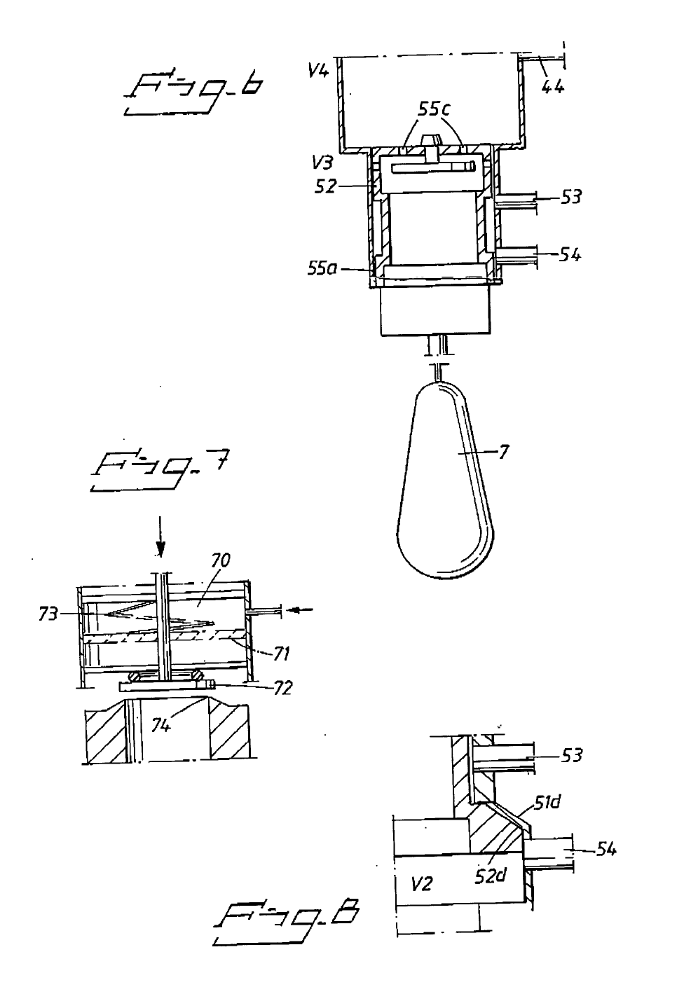

These are not shown in FIGS. 5 to 6, for the sake of clarity, although requisite oblique sealing surfaces in the form of an outer part of the cylinder and an outer part of the piston will be described in more detail with reference to FIG. 8.

The cylinder surface 51a opens via two channels 53, 54, an upper channel, which is used as an air-venting channel 53, and a lower channel, which is used for transferring 54 (48) to the expiration valve 6 an overpressure created in the container 7 or bag.

The channel 54 is thus connected directly with the line 48.

The piston 52 moves vertically up and down in the cylinder 51 and will fall down onto an abutment 51b or said seal under or caused by its own weight, among other things, and takes a lower position. The piston is lifted up to an upper position, by means of gas flowing from beneath.

The lower position, shown in FIG. 6, is taken by the piston during the function mode “CMV” and during that part of the function mode “Spontaneous/Manual” in which manual insufflation is not applied

The piston shall take its upper position, according to FIG. 5, during the manual insufflation phase through the medium of gas flow from said container or bag, so as to conform to the function of the invention.

The piston 52 has a generally thimble-like shape, with an outer cylindrical surface 52a, provided with a peripheral opening 52b adapted to allow an overpressure channel 54 (48) to be connected to the air-venting channel 53 in a lower piston position, a lower position according to FIG. 6.

This arrangement ensures that the cavity 6d of the expiration valve 6 will be evacuated when the piston 52 is in its lower position, and that the surface 6c is subjected to no overpressure that can close the valve 6.

In another position of the piston 52, an upper position according to FIG. 5, the overpressure channel 54 is connected with the line 48 to allow the pressure generated by the container 7 or bag to be transferred to the line 4d and to the expiration valve 6, via a first function mode related valve 49, said valve 6 therewith being kept closed.

This state prevails for as long as a gas flow passes from the container 7 or bag, through the piston 52, and into the hose 44 and in a direction towards the patient 42.

The upper part of the piston 52, or a piston top 52c, co-acts with a one-way valve 55.

The cylindrical part 52a′, close to the piston top 52c, is provided with holes or openings 52a″ so that in an upper position of the piston 52 the reserve volume 7′ is able to pass to the hose 44, as a manually activated insufflation phase.

FIG. 5 shows the valve arrangement 41, belonging to the control unit, in a state in which the reserve volume 7′ in the container 7 or bag can be emptied, either completely or partially, via a manually generated overpressure.

The lower top surface 52c′ of the piston or the piston unit 52 includes a flat one-way valve element 55, that includes a disc-shaped valve body 55b situated within a cavity 52′ in the piston unit 52.

Under the influence of the gas flowing from the container 7 or bag, the valve body 55b is able to move to an upper position or position, in which it seals off a number of holes 55c (see FIG. 6), and is able to drop, under or due to its own weight, to a lower position in which the holes 55c are exposed.

The sealing state (FIG. 5) is taken automatically and initially in response to an overpressure generated from the container 7 or bag, said overpressure and gas flow also lifting the piston unit 52 to an upper position, so as to enable a manual insufflation gas flow to pass from the container 7 or bag to the patient 42, via the valve arrangement 41 and via the hose 44.

FIG. 6 shows the valve arrangement 41 with a piston unit 52 in a downwardly displaced position and in the valve body 55b of the valve device 55 and in a position exposing the holes 55c.

As shown, an empty container 7 or bag (according to FIG. 5) will be successively replenished with a reserve volume 7′ via the valve arrangement 41 and insufflation gas flows passing from the ventilator through the holes 55c and to the container 7 or bag, via the hose 44a.

Thus, the valve arrangement 41 may be considered as having a number of different valve functions, which are all co-ordinated for activation with the aid of the up-and-down movement of the piston 52 in the cylinder 51.

A number of valve functions can be activated in the upper position of the piston 52, according to FIG. 5.

A first valve function “V1” therewith lies in the upward movement of the piston 52, which exposes the holes 52a″ in an upper position for a manual insufflation phase.

A second valve function “V2” lies in the movement of the piston 52, in which the channel 48 is freed from its gas venting function (53) and supplied with overpressure from the container 7 or bag.

A third valve function “V3” lies in the lower position of the piston and when the valve 55 at the piston top is open so that insufflation gas can pass into the container 7 or bag from the hose 44a.

A fourth valve function “V4” is independent of the setting of the piston 52 in the cylinder 51 and its function mode is related as a second function mode related valve.

The valve function “V4” is activated to a first state, a closed state, by the function mode “CMV”, via a remote-controlled device, which is illustrated in FIG. 7 as a pneumatically actuable piston-cylinder arrangement, in which a piston 71 can be moved into contact with a seat 74 by means of a spring 73, so as to provide a sealing function in the function mode “CMV” and moved out of said sealing state in the function mode “Spontaneous/Manual”.

Alternatively, a valve body 72 can be actuated in a similar manner with the aid of an attraction magnet and a spring, and activated to a second state, an open state, in the function mode “Spontaneous/Manual”, according to FIG. 7.

FIG. 8 shows that a seal between the piston 52 and the inner cylindrical surface 51a can be achieved between the surface parts 52d and 51d, which have an oblique shape.

It will be understood that the invention is not restricted to the aforedescribed and illustrated exemplifying embodiment thereof and that modifications can be made within the scope of the inventive concept as illustrated in the accompanying claims.

1. A lung ventilating arrangement comprising; a control unit, a number of gas delivery hoses and a patient-allocated or -related device, wherein first end portions of the hoses face towards the control unit while the other or second end portions of said hoses face towards said device, wherein said control unit includes means for delivering to said patient a gas flow, intended for spontaneous inhalation, and/or a gas flow, intended for insufflation, via a first hose, and wherein an expiration valve, situated close to the patient-allocated device, is adapted to release an expiration gas flow to the free surroundings, and wherein said expiration valve can be moved to one of two settings, via a second of said gas delivery hoses, characterized in that a reserve volume in a container or bag is intended to generate a gas flow, intended for a manual insufflation phase, said gas flow being activated by pressurising the container or bag temporarily and over or during a short time period; in that a reserve volume, in said container or bag, is delivered to the first hose during pressurisation of said container or bag, via a valve arrangement (41); and in that said pressure is delivered to the second hose, via said valve arrangement, so as to cause said expiration valve to take a closed, insufflation phase allocated, state.

2. An arrangement according to claim 1, characterized in that said expiration valve includes a membrane, wherein a pressure-actuated surface, that faces towards a first hose, is smaller than a pressure-actuated surface, that is influenced by the overpressure prevailing in a second hose.

3. An arrangement according to claim 1, characterized in that said expiration valve includes a membrane, whose surface, that faces towards a first hose, is smaller than a surface, that faces towards a cavity in an expiration valve.

4. An arrangement according to claim 1, characterized in that said expiration valve is connected to a patient-allocated or -related device or said device is integral therewith.

5. An arrangement according to claim 1, characterized in that a control unit includes a T-coupling, which belongs to said insufflation hose and to which said container or bag is connected.

6. An arrangement according to claim 1, characterized in that said control unit includes a valve arrangement (41), which allows insufflation gas to pass to the patient as a result of said container or bag related overpressure, and which deflects an overpressure for actuation of said expiration valve.

7. An arrangement according to claim 1, characterized in that said control unit includes a valve unit (49), which is associated with said second gas delivery hose and which is adapted to pressurise said second hose so as to close the expiration valve.

8. An arrangement according to claim 1, characterized in that when in a first state (FIG. 6) said valve arrangement functions to permit the passage of gas flow to the container or bag.

9. An arrangement according to claim 1, characterized in that said arrangement (41) is adapted to include a plurality of valve functions.

10. An arrangement according to claim 6, characterized in that said valve unit (49), associated with said control unit, is actuated by the choice of a prevailing function mode.

11. An arrangement according to claim 1, characterized in that said arrangement includes a cupped membrane, which provides a valve function.

12. An arrangement according to claim 1, characterized in that said valve arrangement includes a first valve function, which is intended to expose piston-associated holes (52″) in the performance of a manual insufflation phase.

13. An arrangement according to claim 1, characterized in that said valve arrangement includes a second valve function, which is adapted for connecting a channel (48) to an overpressure generated in said container or bag.

14. An arrangement according to claim 13, characterized in that said channel is released from a gas evacuating state.

15. An arrangement according to claim 1, characterized in that said valve arrangement includes a third valve function, that is operative adjacent a part of the piston cavity close to the piston top.

16. An arrangement according to claim 15, characterized in that said valve function includes a valve body, which, when in an upper position, can seal against holes that face towards the piston top, and which opens said holes in a lower position.

17. An arrangement according to claim 15, characterized in that insufflation gas is able to pass into the container or bag, when the valve body is in a state in which the holes are open.

18. An arrangement according to claim 1, characterized in that said valve arrangement includes a cylinder and a piston, which is movable in said cylinder, wherein the piston is caused to move to an upper position, by a through-passing gas flow, and to move to a lower position, when the gas flow ceases.

19. An arrangement according to claim 1, characterized in that the valve arrangement includes a fourth valve function, that can be actuated to a first state in a function mode “CMV” and which can be actuated to a second state in a function mode “Spontaneous/Manual”.

20. An arrangement according to claim 19, characterized in that said first state is a state in which the valve closes said container or bag.

21. An arrangement according to claim 1, characterized by an oblique sealing device, that acts between a piston and a cylinder in said valve arrangement.
